(19) United States
(12) Patent Application Publication
(10) Pub. No.: US 2004/0170966 A1
(43) Pub. Date: Sep. 2, 2004

(54) Continuous time-resolved resonance energy-transfer assay for polynucleic acid polymerases

(76) Inventors: Eric Steven Furfine, Durham, NC (US); David John, Timothy Porter, Raleigh, NC (US); Grace Brashear Roberts, Efland, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 09/725,652

(22) Filed: Nov. 29, 2000

Related U.S. Application Data
(60) Provisional application No. 60/167,940, filed on Nov. 29, 1999.

(51) Int. Cl. C12Q 1/68

(52) U.S. Cl. 435/6

(57) ABSTRACT

A method of detecting polynucleic acid polymerase activity, including DNA and RNA polymerase activity. The method includes providing a polynucleic acid primer-template complex labeled with a energy-emitting chemical species and a nucleotide labeled with a energy-emitting chemical species; mixing the polynucleic acid primer-template complex and the nucleotide with a sample comprising or suspected to comprise a polynucleic acid polymerase; prior to, contemporaneously with or after the mixing, exposing the labeled polynucleic acid primer-template complex and the labeled nucleotide to radiation of excitation wavelength for one of the energy-emitting chemical species to thereby excite that energy-emitting chemical species; and detecting a signal produced by energy transfer between the excited energy-emitting chemical species and the other energy-emitting chemical species as a result of incorporation of the nucleotide into the polynucleic acid primer-template complex via the activity of the polynucleic acid polymerase, the detection of the signal indicating polynucleic acid polymerase activity in the sample. Candidate compounds can also be identified as modulators of polynucleic acid polymerase activity via the method.

### CROSS REFERENCE TO RELATED APPLICATIONS

This application is based on and claims priority to U.S. provisional patent application serial No. 60/167,940, filed Nov. 29, 1999, herein incorporated by reference in its entirety.

### TECHNICAL FIELD

The present invention pertains generally to methods of detecting polynucleic acid polymerase activity. More particularly, the present invention pertains to a continuous assay method for detecting polynucleic acid polymerase activity over a predetermined time period and to an assay method for identifying a candidate compound as a modulator of polynucleic acid polymerase activity.

### TABLE OF ABBREVIATIONS

AIDS—acquired immune deficiency syndrome

CY2—a commercially available fluorescent dye

CY3—a commercially available fluorescent dye

CY5—a commercially available fluorescent dye

CY7—a commercially available fluorescent dye

DEPC—diethyl pyrocarbonate

DMSO—dimethyl sulfoxide

DTT—dithiothreitol

em—emission wavelength

ex—excitation wavelength

HIV—human immunodeficiency virus

HTRF—homogeneous time-resolved fluorescence

[I]—inhibitor concentration (units: M)

IC₅₀—concentration in M of modulator which there is 50% modulation of polynucleic acid polymerase activity—the lower the IC₅₀ is, then the more potent the modulator is

infrared 40—a commercially available fluorescent dye

IRD 40—a commercially available fluorescent dye

kₒₙ—on rate constant for modulator binding to polynucleic acid polymerase (units: M⁻¹ min⁻¹)

k_{off}—off rate constant for modulator dissociation from modulator-polynucleic acid polymerase complex (units: min⁻¹)

M—molarity (units: moles/liter)

MR 200—a commercially available fluorescent dye

NNRTI—non-nucleoside reverse transcriptase inhibitor

NP40—Nonidet-P40

RT—reverse transcriptase

SPA—scintillation proximity assay

Vₘₐₓ—uninhibited reaction rate

WT—wild type

### BACKGROUND ART

Polynucleic acid polymerases, including DNA and RNA polymerases, catalyze the incorporation of nucleotides onto template strands of polynucleic acids in vivo. These polymerases thus play important roles in the synthesis of new DNA molecules and in the synthesis of RNA molecules for subsequent translation into functional and structural proteins.

A polynucleic acid polymerase of particular interest is the reverse transcriptase encoded by the human immunodeficiency virus (HIV), the causative agent of acquired immune deficiency syndrome (AIDS). Reverse transcriptase (RT) is essential to viral replication and proliferation. The polymerase is called reverse transcriptase because it catalyzes the synthesis of DNA molecules from the RNA molecules carried by HIV. Thus, this polynucleic acid polymerase, as well as other polynucleic acid polymerases, has been the target of substantial research efforts for modulators of their biological activity, including particularly inhibitors of their biological activity.

For example, non-nucleoside reverse transcriptase inhibitors (NNRTIs) have been identified and are effective in treating AIDS when combined with nucleoside RT inhibitors and HIV protease inhibitors. See Artico, M. (1996), Farmaco 51:305-331; DeClerk, E. (1996), Medical Virology 6:97-117. However, many NNRTIs are slow-binding inhibitors of wild type RT. Determining true affinities of inhibitors requires monitoring the time-course of enzymatic activity. Current methods for measuring RT activity are typically based on radioactive endpoint assays. In such assays, multiple reaction wells that each represent a single time-point must be employed. Thus, monitoring the time-course is a relatively tedious process. Additionally, because many NNRTIs are slow, time-dependent inhibitors of wild type RT, IC₅₀ values determined by conventional endpoint assay methods can be erroneously high.

RT scintillation proximity assay (SPA) currently available from Amersham Life Science, Piscataway, N.J. detects incorporation of (³H)-TMP into a primer-template complex via streptavidin-coated SPA bead that is attached to a 5′-biotin on the primer. The beads must be added to the sample at the end of the reaction because RT cannot efficiently catalyze primer extension in the presence of the beads. Thus, this assay is also effectively an endpoint assay.

What is needed, then, is an assay to monitor the time-course of RT or other polynucleic acid polymerase modulation by NNRTIs or by other candidate modulator compounds. Such an assay would facilitate determination of whether a modulator binds a polynucleic acid polymerase rapidly or slowly; would facilitate calculation of accurate IC₅₀ values; and would allow for relevant comparison of modulation potency between candidate modulators. Such an assay is not currently available in the art.

### SUMMARY OF THE INVENTION

A method of detecting polynucleic acid polymerase activity is disclosed. The method comprises providing a polynucleic acid primer-template complex labeled with an energy-emitting chemical species and a nucleotide labeled with an energy-emitting chemical species; mixing the polynucleic acid primer-template complex and the nucleotide with a sample comprising or suspected to comprise a polynucleic acid polymerase; prior to, contemporaneously with or after the mixing, exposing the labeled polynucleic acid primer-template complex and the labeled nucleotide to radiation of excitation wavelength for one of the energy-emitting chemical species to thereby excite that energy-emitting chemical species; and detecting a signal produced by energy transfer between the excited energy-emitting chemical species and the other energy-emitting chemical species as a result of incorporation of the nucleotide into the polynucleic acid primer-template complex via the activity of the polynucleic acid polymerase, the detection of the signal indicating polynucleic acid polymerase activity in the sample.

A method for identifying a candidate compound as a modulator of polynucleic acid polymerase activity is also disclosed. The method comprises providing a candidate compound, a polynucleic acid primer-template complex labeled with an energy-emitting chemical species and a nucleotide labeled with an energy-emitting chemical species; mixing the candidate compound, the polynucleic acid primer-template complex and the nucleotide with a polynucleic acid polymerase; prior to, contemporaneously with or after the mixing, exposing the labeled polynucleic acid primer-template complex and the labeled nucleotide to radiation of excitation wavelength for one of the energy-emitting chemical species to thereby excite that energy-emitting chemical species; detecting a signal produced by energy transfer between the excited energy-emitting chemical species and the other energy-emitting chemical species as a result of incorporation of the nucleotide into the polynucleic acid primer-template complex via the activity of the polynucleic acid polymerase, the detected signal indicating an amount of polynucleic acid polymerase activity; and identifying the candidate compound as a modulator of polynucleic acid polymerase activity based on the amount of signal detected as compared to a control sample.

Accordingly, it is an object of the present invention to provide a novel assay for polynucleic acid polymerase activity. The object is achieved in whole or in part by the present invention.

An object of the invention having been stated hereinabove, other objects will become evident as the description proceeds when taken in connection with the accompanying Laboratory Examples as best described herein below.

### DETAILED DESCRIPTION OF THE INVENTION

The present invention pertains to a continuous assay for polynucleic polymerase activity that monitors polynucleic acid primer extension based on time-resolved resonance energy transfer, and preferably time-resolved fluorescence energy transfer. The terms “continuous” or “kinetic” are meant to refer to the detection of a signal at a plurality of time points in a single reaction. The present invention thus represents a novel application of the resonance energy transfer that occurs when energy from an excited donor energy-emitting chemical species (e.g. a fluorophore) is transferred directly to an acceptor energy-emitting chemical species (e.g. a fluorophore) in a continuous or kinetic assay for polynucleic acid polymerase activity.

Following long-standing patent law convention, the terms “a” and “an” mean “one or more” when used in this application, including the claims.

Time-resolved, or time-gated fluorescence spectroscopy is described in U.S. Pat. Nos. 4,058,732 and 4,374,120, incorporated by reference herein. This technique employs a fluorescent probe that has a fluorescence decay (lifetime) that substantially exceeds the duration of the exciting pulse and the duration of the background non-specific fluorescence. A time-gating is used to reduce the background fluorescence, i.e., the measurement of the fluorescence is delayed until a certain time has elapsed from the moment of excitation. The delay time is sufficiently long for the background fluorescence to have ceased. When the fluorescence signal is measured (after the delay) the measurement is an integrated measurement, i.e. all the light arriving at the detector during the measuring period is measured without regard to the time of arrival. The purpose of this delayed measurement is to ensure that only one fluorescence signal reaches the detector during measurement.

In accordance with the present invention, a method of detecting polynucleic acid polymerase activity is provided. In the method, a polynucleic acid primer-template complex labeled with an energy-emitting chemical species is provided. Nucleotides labeled with an energy-emitting chemical species are also provided. The polynucleic acid primer-template complex and the nucleotides are mixed in the presence or suspected presence of a polynucleic acid polymerase. Prior to, in conjunction with or after this mixing, the labeled polynucleic acid primer-template complex and the labeled nucleotide are exposed to radiation of excitation wavelength (e.g. with a light pulse) for one of the energy emitting chemical species to thereby excite that energy-emitting chemical species. A signal is produced by energy transfer between the excited energy-emitting chemical species and the other energy-emitting chemical species as a result of incorporation of the nucleotide into the polynucleic acid primer of the polynucleic acid primer-template complex (also referred to herein as “primer extension”) via the activity of the polynucleic acid polymerase. Thus, the detection of the signal indicates the presence of polynucleic acid polymerase activity. Preferably, the signal is detected at a plurality of time points over a predetermined time-period to thereby determine polymerase activity over the predetermined time-period.

The polynucleic acid primer-template complex is prepared by annealing a polynucleic acid primer (e.g. a DNA or an RNA molecule) to a complementary polynucleic acid template (e.g. a DNA or an RNA molecule) under suitable annealing conditions. Representative annealing conditions are provided in the Laboratory Examples herein. Generally, conditions for annealing polynucleic acids are known in the art, see e.g. Sambrook, J., et al., Molecular Cloning: a Laboratory Manual, Cold Spring Harbor, New York, N.Y. (1989), incorporated by reference herein. Any desired polynucleic acid primer-template complex can be employed in accordance with the present invention. With the appropriate primer-template complex, the present invention can be used to measure primer extension catalyzed by polynucleic acid polymerases from any organism, including but not limited to, viruses, bacteria (e.g., E. coli), plants, or animals (mammals).

The term “nucleotide” is believed to be well-understood in the art and is meant to refer to a phosphate ester of a nucleoside, and preferably, to 5′ triphosphate esters of the five major bases of DNA and RNA. The term “nucleotide” therefore includes deoxyribonucleoside triphosphates (dNTP's), e.g. dUTP, dTTP, dATP, dCTP, dGTP, and ribonucleoside triphosphates (NTP's), e.g. ATP, CTP, UTP and GTP. The dNTP's and NTP's can be labeled with an energy-emitting chemical species for use in the method of the present invention. Modified nucleotide bases (e.g. methylated bases) are also contemplated.

Nucleoside triphosphates are substrates for polymerases, and once incorporated, the nucleotide is in the monophosphate form. Thus, the term “nucleotide” as used herein and in the claims is also meant to refer to nucleoside monophosphate molecules. The term “nucleoside monophosphate” includes deoxyribonucleoside monophosphates (dNMP's), e.g. dUMP, dTMP, dAMP, dCMP, dGMP, and ribonucleoside monophosphates (NMP's), e.g. AMP, CMP, UMP and GMP.

The polynucleic acid primer-template complex and the nucleoside triphosphate molecules are conjugated, bound or otherwise labeled with an energy-emitting chemical species as described herein. As used herein, the terms “label” or “labeled” refers to incorporation of an energy-emitting chemical species, e.g., by incorporation into the polynucleic acid primer-template complex of a nucleotide having biotinyl moieties that can be detected by marked avidin (e.g., streptavidin containing a fluorescent marker). Various other methods of labeling polynucleic acids and nucleotides are known in the art and can also be used.

In accordance with the present invention, the detectable signal is generated from resonant interaction between two energy emitting chemical species: an energy contributing donor chemical species and an energy receiving acceptor chemical species. The polynucleic acid primer-template complex can be labeled with the donor chemical species while the nucleoside triphosphate can be labeled with the acceptor chemical species, and vice versa. Within the polynucleic acid primer-template complex, the polynucleic acid primer can be labeled at its 5′ end or the polynucleic acid template can be labeled at its 3′ end or its 5′ end. In either case, in accordance with the biological activity of polynucleic acid polymerases, the labeled nucleotides are incorporated into the 3′ end of the primer to provide the appropriate spatial relationship for resonance energy transfer between the energy-emitting chemical species as disclosed herein. Moreover, in a preferred embodiment of the present invention as disclosed in the Examples presented herein and in accordance with the biological activity of polynucleic acid polymerases, the labeled nucleotide is complementary to the nucleotide base available on the template for primer extension.

The term “energy-emitting chemical species” is believed to be well understood by one of skill in the art and is meant to refer to any chemical species, whether an atom, molecule, complex or other chemical species, that emits energy in response to a stimulus. The methods of the present invention are contemplated to be useful for any combinations of energy-emitting chemical species so long as the emitted energy from one chemical species is sufficiently intense so as to produce as an energy emission from the other chemical species in accordance with the present invention. For example, energy transfer can occur when the emission spectrum of the donor overlaps the absorption spectrum of the acceptor. Thus, in a preferred embodiment of the present invention, acceptor and donor chemical species are chosen and paired together based on these characteristics. Also, the donor and the acceptor must be within a certain distance, i.e. preferably within the same polynucleic acid primer-template complex, from each other.

Preferred “energy-emitting chemical species” comprise luminescent or light emitting molecules, such as fluorescent, phosphorescent, and chemiluminescent molecules, which emit light when excited by excitation light. Preferred donor/acceptor combinations that can be used in the present inventive method are fluorescent donors with fluorescent or phosphorescent acceptors, or phosphorescent donors with phosphorescent or fluorescent acceptors.

Fluorescent compounds can thus be used to label the polynucleic primer-template complexes and nucleotides employed in the methods of the present invention. Representative fluorescent labeling compounds include dinitrophenyl, fluorescein and derivatives thereof (such as fluorescein isothiocyanate), rhodamine, derivatives of rhodamine (such as methylrhodamine and tetramethylrhodamine), phycoerythrin, phycocyanin, allophycocyanin, o-phthaldehyde and fluorescamine. Representative fluorescent dyes include Texas red, Rhodamine green, Oregon green, Cascade blue, phycoerythrin, CY3, CY5, CY2, CY7, coumarin, infrared 40, MR 200, and IRD 40. Representative chemiluminescent labeling compounds are luminol, isoluminol, theromatic acridinium ester, imidazole, acridinium salt and oxalate ester, while representative bioluminescent compounds for purposes of labeling are luciferin, luciferase and aequorin. All of the compounds are available from commercial sources, such as Molecular Probes, Inc., Eugene, Oreg. and Sigma Chemical Company, St. Louis, Mo.

Representative commercially available fluorescent labeled dNTP include fluorescein-dUTP, fluorescein-dATP (Boehringer Mannheim, Indianapolis, Ind.; Pharmacia Biosystems Aktiebolaget, Uppsala, Sweden); Texas red-dCTP and dGTP (NEN-Dupont, Wilmington, Del.), FLUOROLINK™ CY5-dCTP and dUTP as well as FLUOROLINK™ CY3-dCTP and dUTP (Pharmacia Biosystems Aktiebolaget, Uppsala, Sweden) and the labeled dUTP's and UTP's sold under the trademarks ALEXA™ and BIODPY® by Molecular Probes, Inc., Eugene, Oreg.

The energy-emitting chemical species can comprise any of the fluorescent rare earth metals. Preferably, the fluorescent rare earth metal is of the Lanthanide Series (elements 57-70 of the periodic table). The Lanthanide Series comprises lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb) and lutetium (Lu).

The use of lanthanides is preferred, and the use of lanthanide chelates is more preferred, in view of the long lived fluorescence of lanthanide elements, compared to ordinary fluorescent backgrounds which otherwise tend to overwhelm a genuine signal. For example, the trivalent lanthanide ions Eu³⁺, Tb³⁺, and Sm³⁺ all have fluorescent decay times on the order of milliseconds compared to nanosecond decay times for background fluorescence. By irradiating a reaction sample at the appropriate wavelength and energy level, the fluorescence can be measured at a delayed point in time, after background fluorescence has already decayed, but while the lanthanide specimen is still emitting to facilitate detection of polymerase activity via time-resolved fluorescence spectroscopy.

Thus, in a preferred embodiment of the continuous assay of the present invention, a polynucleic acid polymerase catalyzes the incorporation of a deoxyuridine monophosphate (dUMP) or uridine monophosphate (UMP) analog labeled with a fluorescent dye into a lanthanide chelate-labeled primer-template complex. Primer extension is monitored by the fluorescence energy transfer from the lanthanide to incorporated labeled-dUMP or -UMP.

In a more preferred embodiment of the present invention, RT catalyzes the incorporation of a deoxyuridine monophosphate (dUMP) analog labeled with CY5 dye into a europium (Eu)-labeled primer-template complex. Incorporation of CY5-dUMP into the Eu-primer-template complex is monitored by the fluorescence energy transfer from Eu (excitation 340 nm, emission 620 nm) to CY5-dUMP (excitation 649 nm, emission 670 nm). The signal amplitude change is linearly dependent on enzyme concentration and time.

A method for identifying a candidate compound having an ability to modulate polynucleic acid polymerase activity is also disclosed. A polynucleic acid primer-template complex labeled with an energy-emitting chemical species is provided, as is a nucleoside triphosphate labeled with an energy-emitting chemical species. The candidate compound, the polynucleic acid primer-template complex and the nucleoside triphosphate are then mixed. Prior to, contemporaneously with or after mixing, the labeled polynucleic acid primer-template and the labeled nucleoside triphosphate are exposed to radiation of excitation wavelength (e.g. with a light pulse) for one of the energy emitting chemical species to thereby excite that chemical species.

Prior to, contemporaneously with or after the exposure, a polynucleic acid polymerase is added to the mixture. The production of a signal, e.g. a fluorescence signal, is detected, preferably at a plurality of time points over a predetermined time-period. By a “predetermined time-period”, it is meant any suitable time-period over which the time-course of modulation of a polynucleic acid polymerase by a candidate compound can be established. A representative 40 minute time-period is used to establish the time-courses for RT inhibition by candidate compounds in the Laboratory Examples below.

The signal is produced by energy transfer between the excited energy-emitting chemical species and the other energy-emitting chemical species as a result of incorporation of the nucleotide into the polynucleic acid primer-template complex via the activity of the polynucleic acid polymerase. The candidate compound is identified as a modulator of polynucleic acid polymerase activity based on modulation of signal amplitude in the predetermined time-period relative to a control sample.

The method can further comprise determining whether a candidate modulator compound binds the polynucleic acid polymerase rapidly or slowly. Steady-state IC₅₀ values for the candidate modulator compound can also be calculated, thus further providing a relevant comparison of the modulation potency between compounds. The term “candidate compound” or “candidate substrate” is meant to refer to any compound wherein the characterization of the compound's ability to modulate polynucleic acid polymerase activity is desirable. “Modulate” is intended to mean an increase, decrease, or other alteration of any or all biological activities or properties of a polynucleic acid polymerase. Exemplary candidate compounds or substrates include xenobiotics such as drugs and other therapeutic agents, as well as endobiotics such as steroids, fatty acids and prostaglandins. Non-nucleoside reverse transcriptase inhibitors (NNRTIs), which have been shown to be effective in treating AIDS when combined with nucleoside RT inhibitors and HIV protease inhibitors (ee Artico, M. (1996), Farmaco 51:305-331; DeClerk, E. (1996), Medical Virology 6:97-117, incorporated by reference herein), are particularly contemplated candidate compounds, as are nucleoside analogs.

Because many NNRTIs are slow time-dependent inhibitors of wild type (WT) RT, IC₅₀ values determined by conventional endpoint assays for the identification of NNRTI inhibitors can be erroneously high. Thus, in a preferred embodiment of the present invention, the time-course of RT inhibition by NNRTIs is monitored. With the assay method of the present invention, one can determine whether an inhibitor binds RT rapidly or slowly. Steady-state IC₅₀ values can be calculated from these data and the appropriate model, thus providing a relevant comparison of the inhibition potency between compounds.

The determination of steady-state IC₅₀ values for nevirapine, delavirdine, and efavirenz (commercially available compounds currently marketed as NNRTI's) with wild-type RT and 11 NNRTI-resistant mutants is disclosed in the Laboratory Examples. Association and dissociation rate constants were determined for the slow binding inhibitors. Decreased sensitivity to the NNRTIs was associated with increased values of dissociation rate constants.

As also disclosed in the Laboratory Examples, the method of the present invention can be performed within standard multi-well assay plates as are well known in the art, such as 96-well or 384-well micro-titer plates. Thus, a plurality of candidate compounds can be simultaneously screened for an ability to modulate polynucleic acid polymerase activity within multiple wells of a multi-well plate or via multiple samples on a suitable substrate to provide for high throughput screening of samples in accordance with the present invention. Thus, the present invention provides a polynucleic acid polymerase activity assay that allows for the monitoring of the time-course of the primer extension reaction in a single tube or well, rather than in multiple wells that each represent a single time point, to thereby facilitate the obtaining of kinetic data and the analysis of modulator binding characteristics.

Summarily, the assay method of the present invention simplifies and quickens the kinetic analysis of modulator binding, and allows for the determination of values for association and dissociation rate constants. The primer-template complex can be modified to determine modulation (e.g. inhibitory) constants for nucleoside analogs as well as non-nucleoside polymerase inhibitors. Indeed, the assay method of the present invention has been used to determine steady-state IC₅₀ values for non-nucleoside HIV reverse transcriptase inhibitors, as disclosed in the Laboratory Examples.

### LABORATORY EXAMPLES

The following Laboratory Examples have been included to illustrate preferred modes of the invention. Certain aspects of the following Laboratory Examples are described in terms of techniques and procedures found or contemplated by the present inventors to work well in the practice of the invention. These Laboratory Examples are exemplified through the use of standard laboratory practices of the inventors. In light of the present disclosure and the general level of skill in the art, those of skill will appreciate that the following Laboratory Examples are intended to be exemplary only and that numerous changes, modifications and alterations can be employed without departing from the spirit and scope of the invention.

### MATERIALS AND METHODS USED IN LABORATORY EXAMPLE 1

Expression and purification: DNA encoding wild type and mutant HIV-1 reverse transcriptase (RT) was cloned, expressed and purified by standard techniques, such as those described in Ausubel et al., Current Protocols in Molecular Biology, (J. Wylie & Sons, N.Y.)(1992); Adelman, et al. DNA 2:183 (1983); and Messing et al. Third Cleveland Symposium on Macromolecules and Recombinant DNA, Editor A. Walton, Elsevier, Amsterdam (1981), herein incorporated by reference. Table 1 shows the peptide sequence for wild type RT. The residues (L100→I, K103→N, V106→A, V106→I, V108→I, E138→K, Y181→C, Y188→C, and P236→L) that were mutagenized via conventional site-specific mutagenesis techniques are indicated in bold in the peptide sequence. Table 2 indicates the amino acid changes for each mutant RT.

TABLE 1
Polypeptide Sequence for Wild-Type RT Polypeptide (SEQ ID NO: 1)

| | | | | | |
|---|---|---|---|---|---|
| 1 | PISPIETVPV | KLKPGMDGPK | VKQWPLTEEK | IKALVEICTE | MEKEGKISKI |
| 51 | GPENPYNTPV | FAIKKKDSTK | WRKLVDFREL | NKRTQDFWEV | QLGIPHPAGL¹⁰⁰ |
| 101 | KKK¹⁰³KSV¹⁰⁶TV¹⁰⁸LD | VGDAYFSVPL | DEDFRKYTAF | TIPSINNE¹³⁸TP | GIRYQYNVLP |
| 141 | QGWKGSPAIF | QSSMTKILEP | FRKQNPDIVI | Y¹⁸¹QYMDDLY¹⁸⁸VG | SDLEIGQHRT |
| 191 | KIEELRQHLL | RWGLTTPDKK | HQKEPPFLWM | GYELHP²³⁶DKWT | VQPIVLPEKD |
| 241 | SWTVNDIQKL | VGKLNWASQI | YPGIKVRQLC | KLLRGTKALT | EVIPLTEEAE |
| 291 | LELAENREIL | KEPVHGVYYD | PSKDLIAEIQ | KQGQGQWTYQ | IYQEPFKNLK |
| 341 | TGKYARMRGA | HTNDVKQLTE | AVQKITTESI | VIWGKTPKFK | LPIQKETWET |
| 391 | WWTEYWQATW | IPEWEFVNTP | PLVKLWYQLE | KEPIVGAETF | YVDGAANRET |
| 441 | KLGKAGYVTN | RGRQKWTLT | DTTNQKTELQ | AIYLALQDSG | LEVNIVTDSQ |
| 491 | YALGIIQAQP | DQSESELVNQ | IIEQLIKKEK | VYLAWVPAHK | GIGGNEQVDK |
| 551 | LVSAGIRKVL | | | | |

TABLE 2
RT's Screened In Laboratory Examples

RT sub type
WT
L100I
K103N
V106A
V106I
V108I
E138K
Y181C
Y188C
P236L
Y181C/V106A
Y181C//V108I

Biotinylated Template Primer (25:17 mer): All buffers were made with diethyl pyrocarbonate (DEPC)-treated water and autoclaved. Template primer was made in sterile RNase-free containers. 5′-biotinylated DNA primer, biotin-5′-GTC ATA GCT GTT TCC TG-3′ (SEQ ID NO:2), and the RNA template, 5′-AUU UCA CAC AGG AAA CAG CUA UGA C-3′ (SEQ ID NO:3), were custom synthesized by Oligos Etc., Wilsonville, Oreg. The 5′-biotinylated 17-mer DNA primer (40 nmoles) was mixed with the 25-mer RNA template (20 nmoles) in 1 ml of 10 mM Tris-HCI, pH 8.0, 1 mM MgCl₂. The solution was divided into 9×111 μl samples, heated in a dry bath incubator (Fisher Scientific, Pittsburgh, Pa.) at 92° C. for 5 min., cooled to 40° C. over 4 hrs, and stored at −20° C.

Substrate, Enzyme, and Test Compound Solution Preparation:

Substrate solution and diluted RT were prepared on the day of the assay and stored on ice. Test compounds (100 μM in DMSO in column 1 of a 96-well polypropylene plate) were serially diluted 2-fold into DMSO in column 2 through column 11 of the plate using a BIOMEK® 2000 (Beckman Instruments, Fullerton, Calif.). Column 12 of the plate contained only DMSO. The DMSO solutions (10 μl) were then diluted with 140 μl H₂O using a RAPIDPLATE® 96-well pipetting station (Zymark Corporation, Hopkinton, Mass.).

Reagents and Labware.

96 well plates: Polypropylene for intermediate dilutions (Costar, Oneonta, N.Y., catalog #3794) and black round-bottom plates (Dynex Technologies, Chantilly, Va., catalog #7205) for assays.

Assay Buffer: 66.7 mM Tris-HCl, pH 8,107 mM KCI, 13.3 mM MgCl₂, 0.0043% NP40, 13.3 mM DTT.

Cy5-AP3-dUTP: Amersham Life Science, Arlington Heights, Ill., Cat. No. PA55022.

Eu-labeled Streptavidin: Wallac, Gaithersburg, Md., #CR28-100.

RT: (diluted to 5 nM in Assay Buffer);

Substrate Solution: 200 nM Cy5-dUTP, 80 nM Eu-labeled Streptavidin, 80 nM biotinylated template primer in Assay Buffer.

### LABORATORY EXAMPLE 1—RT ASSAY

Reactions contained 100 nM Cy5-dUTP, 40 nM Eu-labeled template-primer complex, 1 nM RT, 47 mM Tris-HCl, 75 mM KCl, 9.3 mM MgCl₂, 0.003% NP40, 9.3 mM dithiothreitol (DTT), and 2% dimethyl sulfoxide (DMSO). Test compound or control solvent (15 μl) was added to each well containing 25 μl of substrate solution. Wells in column 12 contained substrate solution and control solvent without inhibitor and served as uninhibited controls. The Eu chemical species was then excited by exposing the reactions to radiation of excitation wavelength 340 nm with a light pulse.

The assay was initiated by adding 10 μl of diluted RT (wild type RT and RT mutants described above) to each well using a RAPIDPLATE® 96 well pipetting station. The amplitude of the signal was linearly dependent on enzyme concentration and time. Incorporation of Cy5-dUMP into the Eu-labeled template primer (fluorescence energy transfer from Eu (excitation 340 nm, emission 620 nm) to cy5-dUMP (excitation 649 nm, emission 670 nm)) was monitored over 40 minutes by time-resolved fluorescence with a VICTOR²-1420™ Multilabel Counter (Wallac, Gaithersburg, Md.).

### LABORATORY EXAMPLE 2—DATA ANALYSIS

All data reduction was done with scientific graphing and statistical analysis software sold under the registered trademark SigmaPlot® by Jandel Scientific, Corte Madera, Calif. Background fluorescence was subtracted from all fluorescence readings. Data analyses were based on the following scheme:

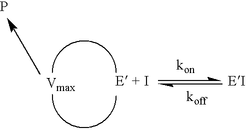

where E′ is a mixture of free enzyme, enzyme-nucleotide complex, enzyme-template primer complex, and enzyme-nucleotide-template primer complex, I is inhibitor, kₒₙ is the inhibitor on rate constant, k_{off} is the inhibitor off rate constant, Vₘₐₓ is the uninhibited reaction rate, and P is the product.

If reactions were linear over 40 min, then IC₅₀ values were determined by fitting equation (1):

y=Vₘₐₓ*IC₅₀*t/(IC₅₀+[I])

to the data where y was the observed fluorescence at time t (minutes), Vₘₐₓ was the uninhibited rate (fluorescence min⁻¹), and [I] was the inhibitor concentration (molar, or M).

If inhibited reactions were not linear over 40 min, indicating slow time-dependent inhibition, values of kinetic constants kₒₙ and k_{off} were determined by non-linear lease square fit of the equation (2):

y=(Vₘₐₓ*k_{off}/(kₒₙ*[I]+k_{off}))*t)+(Vₘₐₓ*kₒₙ*[I]/((kₒₙ*[I]+k_{off})\hat{0}2))*(1−exp(−(kₒₙ*[I]+k_{off})*t)

where y, Vₘₐₓ, and I were defined as above, k_{off} was the off rate constant (min⁻¹), and kₒₙ was the on rate constant (M⁻¹ min⁻¹). The IC₅₀ value was determined by equation (3):

IC₅₀=kₒₙ/k_{off}(M).

TABLE 3
Assay Results

| | nevirapine | | | delavirdine | | | efavirenz | | |
|---|---|---|---|---|---|---|---|---|---|
| | | k_{off} | IC₅₀ | kₒₙ | k_{off} | IC₅₀ | kₒₙ | k_{off} | IC₅₀ |
| RT | kₒₙ (M⁻¹ min⁻¹) | (min⁻¹) | (M) | (M⁻¹ min⁻¹) | (min⁻¹) | (M) | (M⁻¹ min⁻¹) | (min⁻¹) | (M) |
| WT | 2.94 × 10⁵ | 5.61 × 10⁻² | 1.91 × 10⁻⁷ | 1.08 × 10⁶ | 3.78 × 10⁻² | 3.50 × 10⁻⁸ | 1.78 × 10⁷ | 1.48 × 10⁻² | <3.00 × 10^{-9 3} |
| L100I | na¹ | na | 1.51 × 10⁻⁶ | na | na | 1.70 × 10⁻⁶ | 2.31 × 10⁷ | 4.11 × 10⁻¹ | 1.84 × 10⁻⁸ |
| K103N | na | na | 1.12 × 10⁻⁵ | na | na | 9.12 × 10⁻⁷ | na | na | 2.85 × 10⁻⁸ |
| V106A | na | na | 5.01 × 10⁻⁵ | 1.24 × 10⁶ | 2.73 × 10⁻¹ | 2.20 × 10⁻⁷ | 1.29 × 10⁷ | 3.45 × 10⁻² | 2.66 × 10⁻⁹ |
| V106I | nt² | nt | nt | 1.45 × 10⁶ | 4.78 × 10⁻² | 3.30 × 10⁻⁸ | 1.34 × 10⁷ | 1.41 × 10⁻² | <3.00 × 10^{-9 3} |
| V108I | na | na | 6.76 × 10⁻⁶ | 3.34 × 10⁶ | 1.20 × 10⁻¹ | 3.59 × 10⁻⁸ | 1.94 × 10⁷ | 3.03 × 10⁻² | <3.00 × 10^{-9 3} |
| E138K | 1.62 × 10⁵ | 6.62 × 10⁻² | 4.09 × 10⁻⁷ | 1.27 × 10⁶ | 3.97 × 10⁻² | 3.13 × 10⁻⁸ | 6.62 × 10⁶ | 1.35 × 10⁻² | <3.00 × 10^{-9 3} |
| Y181C | na | na | 3.47 × 10⁻⁵ | na | na | 7.59 × 10⁻⁷ | 8.80 × 10⁶ | 1.70 × 10⁻² | <3.00 × 10^{-9 3} |
| Y181C/V106A | na | na | >2.00 × 10⁻⁶ | na | na | >2.00 × 10⁻⁶ | 2.35 × 10⁷ | 1.33 × 10⁻¹ | 5.63 × 10⁻⁹ |
| Y181C/V108I | na | na | >2.00 × 10⁻⁶ | na | na | 7.59 × 10⁻⁷ | 2.22 × 10⁷ | 7.94 × 10⁻² | 3.60 × 10⁻⁹ |
| Y188C | na | na | >2.51 × 10⁻⁵ | na | na | 1.17 × 10⁻⁷ | 2.66 × 10⁷ | 2.12 × 10⁻¹ | 8.01 × 10⁻⁹ |
| P236L | 1.66 × 10⁵ | 5.79 × 10⁻² | 3.49 × 10⁻⁷ | na | na | 1.26 × 10⁻⁶ | 5.71 × 10⁶ | 8.02 × 10⁻³ | <3.00 × 10^{-9 3} |

¹na - not applicable - Inhibited reaction rates were linear over the course of the reactions. k_{off} > 0.5 min⁻¹.
²nt - not tested.
³Calculated IC₅₀ value was less than three times the enzyme concentration in the reaction (1 nM).

### MATERIALS AND METHODS USED IN LABORATORY EXAMPLE 3

Expression and purification: as described for Laboratory Example 1, except only wild type RT.

Biotinylated Template Prime (25:17 mer): All buffers were made with diethyl pyrocarbonate (DEPC)-treated water and autoclaved. Template primer was made in sterile RNase-free containers. 5′-biotinylated DNA primer, biotin5′-GTC ATA GCT GTT TCC TG-3′ (SEQ ID NO:2), and the RNA template, 5′-AUU UCA CAC AGG AAA CAG CUA UGA C-3′ (SEQ ID NO:3), were custom synthesized by Oligos Etc., Wilsonville, Oreg. The 5′-biotinylated 17-mer DNA primer (40 nmoles) was mixed with the 25-mer RNA template (20 nmoles) in 1 ml of 10 mM Tris-HCI, pH 8.0, 1 mM MgCl₂. The solution was divided into 9×111 μl samples, heated in a dry bath incubator (Fisher Scientific, Pittsburgh, Pa.) at 92° C. for 5 min., cooled to 40° C. over 4 hrs, and stored at −20° C.

Substrate, Enzyme, and Text Compounds Solution Preparation:

Substrate solution and diluted RT were prepared on the day of the assay. Substrate solution was maintained at 4° C. One microliter of test compounds (0.5 mM in DMSO) were dispensed in columns 1 through 20 of 384 well plates, and one microliter of DMSO was dispensed in column 21.

Reagents and Labware,

384 well plates: Costar 384 well assay plates, solid black, #3710

Assay buffer: 50 mM Tris-HCI pH 8.0, 80 mM KCl, 10 mM MgCl₂, 0.0032% NP40, 10 mM L-cysteine.

Biotinylated Template Prime (25:17 mer): as described for Laboratory Example 1 above.

Cy5-AP3-dUTP: as described in Laboratory Example 1 above.

RT: diluted to 1.25 nM in Assay Buffer.

Substrate Solutions: 100 nM Cy5-dUTP, 20 nM Eu-labeled Streptavidin, 20 nM biotinylated template primer in Assay Buffer

Thymidine triphosphate (TTP): 9 micromolar Tris Hydrochloride solution

### LABORATORY EXAMPLE 3—HIGH THROUGHPUT RT ASSAY

RT Assay: Reactions contained 20 nM Cy5-dUTP, 4 nM Eu-labeled template primer, 1 nM RT, 50 mM Tris-HCl, 80 mM KCl, 10 mM MgCl2, 0.0032% NP40, 10 mM L-Cysteine, 2% DMSO and 1 nM RT. Stock substrate and TTP solutions were maintained at 4° C. throughout the assay. RT was kept at ambient temperature. Using a BIOMEK® 2000, 10 μl of substrate solution were added to each well containing 1 μl test compound or DMSO. TTP (10 μl 9 μM) was added to wells I21-P21 prior to the start of the reaction to inhibit any Cy5-dUMP incorporation into the primer template. These wells served as background controls. Wells A21-H21 contained DMSO only and served as uninhibited controls. Serially diluted positive controls with known inhibitors were also included on separate wells. Columns 22-24 were empty on both test and control plates.

The RT reactions were initiated by the addition of 40 μl of dilute RT to each well using a MULTIDROP™ 384 (available from Titertek Instruments, Inc. of Huntsville, Ala.) and incubated at ambient temperature. The rate of Cy5-dUMP incorporation into the Eu-labeled template primer was determined by measuring time-resolved fluorescence at approximately 5 minutes and 40 minutes after enzyme addition with a VICTOR™ 1420 Multilabel Counter (Wallac, Gaithersburg, Md.).

### LABORATORY EXAMPLE 4—DATA ANALYSIS FOR HIGH THROUGHPUT ASSAY

The rate of Cy5dUMP incorporation was calculated by subtracting the time-resolved florescence measured 5 minutes after enzyme addition from the time-resolved fluorescence measured at 40 min and dividing by the time interval. The results for each test well in the primary screen were expressed as % inhibition (I) calculated according to the equation (4):

%I=1−x(1−(rateₛₐₘₚₗₑ/rate_{control})

where rateₛₐₘₚₗₑ is the Cy5dUMP incorporation rate in the presence of test compound, and rate_{control} is the rate in the absence of any test compound. The value for rate_{control} was the average of the control wells included in every plate.

For the standard inhibitors, the % control activity (% C) at each concentration of standard inhibitor was calculated by the equation (5):

% C=100×(rateₛₐₘₚₗₑ/rate_{control})

IC₅₀ values for the inhibitors were determined by non-linear least square fit of the equation (6):

% C=Vₘₐₓ×(1−(X/(IC₅₀+X))

to the data, where % C is the activity observed at inhibitor concentration X and Vₘₐₓ is the rate in the absence of inhibitor (˜100%).

IC₅₀ values and percent inhibition reported for NNRTIs are dependent on the template primer used and the time of incubation. Therefore, consistency in assay format is preferred. The rate calculation assumes incorporation is linear over a 35 minute time interval (5 and 40 minutes after enzyme addition) in the presence or absence of inhibitor. This is not the case for slow-binding inhibitors. The IC₅₀ value for a slow-binding inhibitor determined by the 2 time-point method will therefore be higher than that determined by a full inhibition time-course analysis.

The application of which this description and claims form a part can be used as a basis for priority in respect of any subsequent application. The claims of such subsequent application can be directed to any feature or combination of features described herein. They can take the form of product, composition, process or use claims and can include, by way of example and without limitation, one or more of the following claims.

It will be understood that various details of the invention can be changed without departing from the scope of the invention. Furthermore, the foregoing description is for the purpose of illustration only, and not for the purpose of limitation—the invention being defined by the claims.

What is claimed is:

1. A method of detecting polynucleic acid polymerase activity, the method comprising:
(a) providing a polynucleic acid primer-template complex labeled with an energy-emitting chemical species and a nucleotide labeled with an energy-emitting chemical species;
(b) mixing the polynucleic acid primer-template complex and the nucleotide with a sample comprising or suspected to comprise a polynucleic acid polymerase;
(c) prior to, contemporaneously with or after the mixing of step (b), exposing the labeled polynucleic acid primer-template complex and the labeled nucleotide to radiation of excitation wavelength for one of the energy-emitting chemical species to thereby excite that energy-emitting chemical species; and
(d) detecting a signal produced by energy transfer between the excited energy-emitting chemical species and the other energy-emitting chemical species as a result of incorporation of the nucleotide into the polynucleic acid primer-template complex via the activity of the polynucleic acid polymerase, the detection of the signal indicating polynucleic acid polymerase activity in the sample.

2. The method of claim 1, wherein the nucleotide is selected from the group consisting of dUTP, dTTP, dATP, dCTP, dGTP, ATP, CTP, UTP, GTP and combinations thereof.

3. The method of claim 1, wherein the energy-emitting chemical species on the polynucleic acid primer-template complex is a donor chemical species and the energy-emitting chemical species on the nucleotide is an acceptor chemical species or wherein the energy-emitting chemical species on the nucleotide is a donor chemical species and the energy-emitting chemical species on the polynucleic acid primer-template complex is an acceptor chemical species.

4. The method of claim 1, wherein the energy-emitting chemical species on the polynucleic acid primer-template complex and the energy-emitting chemical species on the nucleotide are light-emitting chemical species.

5. The method of claim 4, wherein the light-emitting chemical species are each selected from the group consisting of a fluorescent compound, a phosphorescent compound, a chemiluminescent compound, and a bioluminescent compound.

6. The method of claim 5, wherein the fluorescent compound is selected from the group consisting of fluorescein and derivatives thereof, rhodamine and derivatives thereof, phycoerythrin, phycocyanin, allophycocyanin, o-phthaldehyde, fluorescamine, Texas red, cascade blue, Oregon green, phycoerythrin, CY3, CY5, CY2, CY7, coumarin, infrared 40, MR 200 and IRD 40.

7. The method of claim 5, wherein the light-emitting chemical species on the polynucleic acid primer-template complex, the light-emitting chemical species on the nucleotide or both the light-emitting chemical species on the polynucleic acid primer-template complex and the light-emitting chemical species on the nucleotide are rare earth metals.

8. The method of claim 7, wherein the rare earth metal light-emitting chemical species on the polynucleic acid primer-template complex, the rare earth metal light-emitting chemical species on the nucleotide or both the rare earth metal light-emitting chemical species on the polynucleic acid primer-template complex and the rare earth metal light-emitting chemical species on the nucleotide are lanthanides.

9. The method of claim 8, wherein the lanthanide further comprises a lanthanide chelate.

10. The method of claim 9, wherein the lanthanide chelate further comprises lanthanum, cerium, praseodymium, neodymium, promethium, samarium, europium, gadolinium, terbium, dysprosium, holmium, erbium, thulium, ytterbium or lutetium.

11. The method of claim 5, wherein the chemiluminescent compound is selected from the group consisting of luminol, isoluminol, theromatic acridinium ester and acridinium salt.

12. The method of claim 5, wherein the bioluminescent compound is selected from the group consisting of luciferin, luciferase and aequorin.

13. The method of claim 1, wherein the polynucleic acid polymerase is a DNA polymerase or a RNA polymerase.

14. The method of claim 13, wherein the polymerase is a reverse transcriptase.

15. The method of claim 1, further comprising detecting the signal at a plurality of time points over a predetermined time-period.

16. The method of claim 1, further comprising screening a plurality of samples simultaneously for polynucleic acid polymerase activity.

17. The method of claim 16, wherein steps (a) through (d) are carried out for each sample in a single well of a multi-well plate.

18. A method for identifying a candidate compound as a modulator of polynucleic acid polymerase activity, the method comprising:
(a) providing a candidate compound, a polynucleic acid primer-template complex labeled with an energy-emitting chemical species and a nucleotide labeled with an energy-emitting chemical species;
(b) mixing the candidate compound, the polynucleic acid primer-template complex and the nucleotide with a polynucleic acid polymerase;
(c) prior to, contemporaneously with or after the mixing of step (b), exposing the labeled polynucleic acid primer-template complex and the labeled nucleotide to radiation of excitation wavelength for one of the energy-emitting chemical species to thereby excite that energy-emitting chemical species;
(d) detecting a signal produced by energy transfer between the excited energy-emitting chemical species and the other energy-emitting chemical species as a result of incorporation of the nucleotide into the polynucleic acid primer-template complex via the activity of the polynucleic acid polymerase, the detected signal indicating an amount of polynucleic acid polymerase activity; and
(e) identifying the candidate compound as a modulator of polynucleic acid polymerase activity based on the amount of signal detected as compared to a control sample.

19. The method of claim 18, wherein the nucleotide is selected from the group consisting of dUTP, dTTP, dATP, dCTP, dGTP, ATP, CTP, UTP, GTP and combinations thereof.

20. The method of claim 18, wherein the energy-emitting chemical species on the polynucleic acid primer-template complex is a donor chemical species and the energy-emitting chemical species on the nucleotide is an acceptor chemical species or wherein the energy-emitting chemical species on the nucleotide is a donor chemical species and the energy-emitting chemical species on the polynucleic acid primer-template complex is an acceptor chemical species.

21. The method of claim 18, wherein the energy-emitting chemical species on the polynucleic acid primer-template complex and the energy-emitting chemical species on the nucleotide are light-emitting chemical species.

22. The method of claim 21, wherein the light-emitting chemical species are each selected from the group consisting of a fluorescent compound, a phosphorescent compound, a chemiluminescent compound, and a bioluminescent compound.

23. The method of claim 22, wherein the fluorescent compound is selected from the group consisting of fluorescein and derivatives thereof, rhodamine and derivatives thereof, phycoerythrin, phycocyanin, allophycocyanin, o-phthaldehyde, fluorescamine, Texas red, cascade blue, Oregon green, phycoerythrin, CY3, CY5, CY2, CY7, coumarin, infrared 40, MR 200 and IRD 40.

24. The method of claim 22, wherein the light-emitting chemical species on the polynucleic acid primer-template complex, the light-emitting chemical species on the nucleotide or both the light-emitting chemical species on the polynucleic acid primer-template complex and the light-emitting chemical species on the nucleotide are rare earth metals.

25. The method of claim 24, wherein the rare earth metal light-emitting chemical species on the polynucleic acid primer-template complex, the rare earth metal light-emitting chemical species on the nucleotide or both the rare earth metal light-emitting chemical species on the polynucleic acid primer-template complex and the rare earth metal light-emitting chemical species on the nucleotide are lanthanides.

26. The method of claim 25, wherein the lanthanide further comprises a lanthanide chelate.

27. The method of claim 26, wherein the lanthanide complex further comprises lanthanum, cerium, praseodymium, neodymium, promethium, samarium, europium, gadolinium, terbium, dysprosium, holmium, erbium, thulium, ytterbium or lutetium.

28. The method of claim 22, wherein the chemiluminescent compound is selected from the group consisting of luminol, isoluminol, theromatic acridinium ester and acridinium salt.

29. The method of claim 22, wherein the bioluminescent compound is selected from the group consisting of luciferin, luciferase and aequorin.

30. The method of claim 18, wherein the polynucleic acid polymerase is a DNA polymerase or a RNA polymerase.

31. The method of claim 30, wherein the polymerase is a reverse transcriptase.

32. The method of claim 18, further comprising detecting the signal at a plurality of time points over a predetermined time period.

33. The method of claim 32, further comprising calculating an association constant and a dissociation constant for the candidate compound for modulation of polynucleic acid polymerase activity.

34. The method of claim 32, further comprising calculating an IC₅₀ value for the candidate compound for modulation of polynucleic acid polymerase activity.

35. The method of claim 18, further comprising screening a plurality of candidate compounds simultaneously for polynucleic acid polymerase modulator activity.

36. The method of claim 35, wherein steps (a) through (d) are carried out for each sample in a single well of a multi-well plate.

37. A method for identifying a candidate compound as a modulator of polynucleic acid polymerase activity, the method comprising:
(a) providing a candidate compound, a polynucleic acid primer-template complex labeled with a light-emitting chemical species and a nucleotide labeled with a light-emitting chemical species;
(b) mixing the candidate compound, the polynucleic acid primer-template complex and the nucleotide with a polynucleic acid polymerase;
(c) prior to, contemporaneously with or after the mixing of step (b), exposing the labeled polynucleic acid primer-template complex and the labeled nucleotide to radiation of excitation wavelength for one of the light-emitting chemical species to thereby excite that light-emitting chemical species;
(d) detecting a signal at a plurality of time points over a predetermined time period, the signal produced by energy transfer between the excited light-emitting chemical species and the other light-emitting chemical species as a result of incorporation of the nucleotide into the polynucleic acid primer-template complex via the activity of the polynucleic acid potymerase, the detected signal indicating an amount of polynucleic acid polymerase activity; and
(e) identifying the candidate compound as a modulator of polynucleic acid polymerase activity based on the amount of signal detected as compared to a control sample.

38. The method of claim 37, wherein the nucleotide is selected from the group consisting of dUTP, dTTP, dATP, dCTP, dGTP, ATP, CTP, UTP, GTP and combinations thereof.

39. The method of claim 37, wherein the light-emitting chemical species on the polynucleic acid primer-template complex is a donor chemical species and the light-emitting chemical species on the nucleotide is an acceptor chemical species or wherein the light-emitting chemical species on the nucleotide is a donor chemical species and the light-emitting chemical species on the polynucleic acid primer-template complex is an acceptor chemical species.

40. The method of claim 37, wherein the light-emitting chemical species are each selected from the group consisting of a fluorescent compound, a phosphorescent compound, a chemiluminescent compound, and a bioluminescent compound.

41. The method of claim 40, wherein the fluorescent compound is selected from the group consisting of fluorescein and derivatives thereof, rhodamine and derivatives thereof, phycoerythrin, phycocyanin, allophycocyanin, o-phthaldehyde, fluorescamine, Texas red, cascade blue, Oregon green, phycoerythrin, CY3, CY5, CY2, CY7, coumarin, infrared 40, MR 200 and IRD 40.

42. The method of claim 40, wherein the light-emitting chemical species on the polynucleic acid primer-template complex, the light-emitting chemical species on the nucleotide or both the light-emitting chemical species on the polynucleic acid primer-template complex and the light-emitting chemical species on the nucleotide are rare earth metals.

43. The method of claim 42, wherein the rare earth metal light-emitting chemical species on the polynucleic acid primer-template complex, the rare earth metal light-emitting chemical species on the nucleotide or both the rare earth metal light-emitting chemical species on the polynucleic acid primer-template complex and the rare earth metal light-emitting chemical species on the nucleotide are lanthanides.

44. The method of claim 43, wherein the lanthanide further comprises a lanthanide chelate.

45. The method of claim 44, wherein the lanthanide comprises lanthanum, cerium, praseodymium, neodymium, promethium, samarium, europium, gadolinium, terbium, dysprosium, holmium, erbium, thulium, ytterbium or lutetium.

46. The method of claim 40, wherein the chemiluminescent compound is selected from the group consisting of luminol, isoluminol, theromatic acridinium ester and acridinium salt.

47. The method of claim 40, wherein the bioluminescent compound is selected from the group consisting of luciferin, luciferase and aequorin.

48. The method of claim 40, wherein the light-emitting chemical species is a lanthanide chelate and the light-emitting chemical species is a fluorescent dye.

49. The method of claim 37, wherein the polynucleic acid polymerase is a DNA polymerase or a RNA polymerase.

50. The method of claim 49, wherein the polymerase is a reverse transcriptase.

51. The method of claim 37, further comprising calculating an association constant and a dissociation constant for the candidate compound for modulation of polynucleic acid polymerase activity.

52. The method of claim 37, further comprising calculating an IC₅₀ value for the candidate compound for modulation of polynucleic acid polymerase activity.

53. The method of claim 37, further comprising screening a plurality of candidate compounds simultaneously for polynucleic acid polymerase modulator activity.

54. The method of claim 53, wherein steps (a) through (d) are carried out for each sample in a single well of a multi-well plate.
